(19) United States
(12) Patent Application Publication
(10) Pub. No.: US 2023/0338605 A1
(43) Pub. Date: Oct. 26, 2023

(54) AN AIR PURIFICATION SYSTEM AND METHOD

(71) Applicant: INDIAN INSTITUTE OF TECHNOLOGY BOMBAY, Mumbai (IN)

(72) Inventors: Ravi KAUSHIK, New delhi (IN); Yelia SHANKARANARAYANA MAYYA, Vashi (IN); Rochish THAOKAR, Mumbai (IN)

(21) Appl. No.: 18/015,276

(22) PCT Filed: Jul. 7, 2021
(86) PCT No.: PCT/IN2021/050664

§ 371 Date: Jan. 9, 2023

(30) Foreign Application Priority Data
Jul. 7, 2020 (IN) 202021028874

(51) Int. Cl. A61L 9/20; B01D 53/00; B01D 53/88; B01D 46/00; B01D 46/10; B01D 46/80; B03C 3/016; B03C 3/155; A61L 9/22; A61L 9/014; F24F 8/167; F24F 8/22; F24F 8/30; F24F 8/90

(52) U.S. Cl. CPC A61L 9/205; B01D 53/007; B01D 53/885; B01D 46/0028; B01D 46/0032; B01D 46/0036; B01D 46/10; B01D 46/80; B03C 3/016; B03C 3/155; B01D 46/0049; A61L 9/22; A61L 9/014; F24F 8/167; F24F 8/22; F24F 8/30; F24F 8/90; B01D 2259/804; B01D 2279/65; B01D 2257/708; B01D 2253/102; B01D 2273/30; B01D 2255/802; B01D 2255/9155; B01D 2255/20707; B01D 2255/20761; B01D 2255/20792; B01D 2255/30; B01D 2257/91; B01D 2258/06; A61L 2209/12; A61L 2209/14; A61L 2209/22

(57) ABSTRACT

The invention relates to the field of air purification, sterilization, disinfection, sanitization, removal of particulate matter and harmful gases. A UV light source(102), which is preferably positioned parallel to the flow of air ensuring continuous disinfection of the airstream due to increased residence time, and a filter (106) that is preferably positioned surrounding/around the UV source. In an embodiment, the invention is disclosing a self-cleaning ability that is achieved by using electrostatic fields. In another embodiment, a small, compact self-cleaning purifier/device can be used to sanitize small, enclosed spaces like cars, cabins, etc.

### FIELD OF THE INVENTION

The invention relates to air purifiers and a method of purification.

More particularly, the invention relates to the field of air purification and disinfection.

### BACKGROUND OF THE INVENTION

The COVID 19 pandemic has played a key role in highlighting the need for sanitization and disinfection practices to control the spread of pathogenic organisms in enclosed environments such as in transportation and business settings/restaurants or residential settings.

For ages, UV light has been used to destroy pathogens in the environment. In recent years, the use of chemical fogging systems to disinfect the environment is also well-known. However, there is a continuous build-up of pathogens and germs on the surface of the existing air sanitizers/filters requiring the same to be shut-off and cleaned at regular intervals, to maintain efficiency.

A PCT publication no. WO2018093837 by Aerobiotix, Inc. dated 15, Nov. 2017, titled “Air germicidal device” discloses an air germicidal treatment device and system for removing or eliminating unwanted pathogens or bacteria in an airstream. The device or system has a removable irradiation chamber that divides an interior area of the device into an air pre-chamber area, an air post-chamber area, and an irradiation area therebetween.

A US patent numbered U.S. Pat. No. 5,185,015 by Searle Bruce R dated 1991 Mar. 18, titled “Filter apparatus” discloses a filter apparatus for removing contaminants from air including a first filter element removing particles greater than a predetermined size from air entering the housing by way of the inlet opening, and a second filter element removing particles of selected chemical species from the air that has passed through the first filter element in the direction from the inlet opening to the outlet opening. The second filter element defines a chamber that is in open communication with the outlet opening, and the chamber constitutes a third filter element in which air is irradiated with ultraviolet light.

However, such purification systems may become a source for growth of microorganisms and dirt, thereby increasing risk of resuspension and reduced efficiency.

In view of the above and other improvement requirements, there is a need to develop a self-cleaning air purifying device or a system and method of purification which removes and kills pathogenic contaminants from the air and also renders it cleaner from particular matter and harmful gases.

### OBJECTS OF THE INVENTION

An object of the present invention is to provide a self-cleaning air purifier and a method of purification which sanitizes, disinfects, and removes airborne pathogens.

Another object of the present invention is to provide a self-cleaning air purifier having a housing (with one inlet and one outlet, airflow means, one filter, at least one UV source, a chemical oxidant system, ionization system and chamber for placing objects and/or belongings therein for disinfection.

Another object of the present invention is to provide a UV light source, which is preferably positioned longitudinally to the flow of air ensuring continuous disinfection of the incoming airstream due to increased residence time. In a setting allowing the air to flow parallel to the surface of the UV source.

Another object of the present invention is to provide a filter that is preferably positioned surrounding/around the UV source. The filter is positioned such that air flows from the inner side to the outer side, where the inner side capturing the impurities are continuously subject to UV radiation and ionization related purifications, such as OH radical formation and inductive and capacitive forces on impurities.

Another object of the present invention is to provide a filter, that is preferably UV resistant, and the inner side of the filter is coated with a photocatalyst.

Another object of the present invention is to provide an inner coating that will enhance the process of disinfection by the production of OH radicals.

Another object of the present invention is to provide a filter that may further have the outer side coated with an adsorbent to trap gases and VOCs (volatile organic compounds).

Another object of the present invention is to provide an outer coating of the photocatalyst that will ensure the surface of the purifier/device/system remains pathogen-free.

Another object of the present invention is to provide a filter that may also be impregnated with antimicrobial coating which may have silver and/or copper nanoparticles for further sanitization and disinfection effect.

Another object of the present invention is to provide a self-cleaning ability that is achieved by using electrostatic fields.

Another object of the present invention is to provide needles of ionizers that are used to create electric fields for air purification.

Another object of the present invention is to provide thin carbon fibers, which operate at a very high voltage and remove all kinds of deposited contaminants from the UV source surface.

Yet another object of the present invention is to provide a chemical oxidant that acts as an on-demand disinfection system.

Yet another object of the present invention is to provide an oxidant, which is stored in a non-reactive container inside the housing with a pump for atomization through the nozzle.

Still another object of the present invention is to provide a disinfecting chamber, which uses UV rays for the disinfection of small objects/items.

Still another object of the present invention is to provide a disinfecting chamber, and the walls of this chamber are coated with a material that reflects maximum UV light.

Still another object of the present invention is to provide an electrical compartment, which gives an appropriate power supply as well as controls the power supply.

Still another object of the present invention is to provide a sensor, which may also be provided such that it detects movement and automatically switches the purifier/device ON/OFF.

Still another object of the present invention is to provide outlet grills/housing, which is designed in such a way that there would avoid any direct eye contact with the UV source.

Still another object of the present invention is to provide a small, compact self-cleaning purifier/device/system that can be used to sanitize small, enclosed spaces like cars, cabins, etc.

Still another object of the present invention is to provide a method of purification and self-cleaning consisting of a plurality of purification mechanisms synergizing to create an increased efficiency.

Still another object of the present invention is to provide a DCD (Deactivate, Capture, Deactivate) approach/mechanism having a plurality of steps for air purification and disinfection.

### SUMMARY OF THE INVENTION

An aspect of the invention is disclosing about an air purification system, for purifying an incoming impure air, the system comprising a UV source configured to produce irradiation to effect microorganisms/pathogens. An ionizer configured to generate dense electric field and thus produce OH radicals and/or H202 using the energy imparted by the UV source and a water source; A filter having an inner surface and an outer surface, configured to trap impurities of the impure air and produce filtered air from the outer surface. The UV source and ionizer are configured to face the inflow of impure air before the impure air reaches the filter. A housing, with at least an inlet, which is placed longitudinally towards the center of the housing, at a specified distance from the bottom; with at least a UV source, which kill microorganisms; with at least an electrical compartment, a photocatalytic coating on inside the filter assembly; with at least a chemical oxidant system, with at least a blower, and with at least an outlet; at least one needle of ionizer and a chamber for placing objects and/or belongings therein for disinfection.

Another aspect of the invention discloses about a UV source, which is positioned longitudinally to the flow of air ensuring continuous disinfection of the airstream due to increased residence time. The UV source has a self-cleaning ability which is achieved by using electrostatic fields.

Another aspect of the invention discloses about a self-cleaning process, which includes carbon fibers from the ionizer which operates at very high voltage to remove all kinds of deposited contaminants from the surface of the UV source.

Another aspect of the invention discloses about a filter, which is placed above the UV source, and the air after irradiation with the UV rays passes through the filter. The filter is positioned such that air flows from the inner side to the outer side. The filter is preferably UV resistant, and the inner side of the filter is coated with a photocatalyst. The filter further has an outer side coated with an adsorbent to trap gases and VOCs (volatile organic compounds).

Yet another aspect of the invention disclosed about a chemical oxidant is placed on the outer side of the filter and acts as on-demand disinfection.

Still, another aspect of the invention disclosed about the method, which consists of a plurality of steps, includes different phases through which air travels and purifies using the DCD (deactivate, Capture, Deactivate) approach/mechanism.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in the context of these embodiments, it should be understood that it is not intended to limit the scope of the invention to these particular embodiments.

FIG. 1 illustrates a self-cleaning air purifier in accordance with an embodiment herein.

FIG. 2 illustrates a self-cleaning air purifier as per an embodiment herein.

FIG. 3 illustrates a view of the self-cleaning air purifier, as per an embodiment herein.

FIG. 4 illustrates another view of the self-cleaning air purifier, as per an embodiment herein.

FIG. 5 illustrates another view of the self-cleaning air purifier, as per an embodiment herein.

FIG. 6 illustrates a self-cleaning air purifier housing, as per an embodiment herein.

FIG. 7 illustrates a method of purification and self-cleaning as per an embodiment herein.

FIG. 8 illustrates a DCD approach/mechanism as per an embodiment herein.

The referral numerals in the figures refer to: (100) an air purifier; (100A)—housing; (101)—inlet; (102)—UV source; (103)—Electrical compartment; (104) Ionizer or plasma generator; (104A)—needle; (104B) Chamber; (105)—Photocatalytic coating; (106)—Filter; (107)—Chemical oxidant system; (107A)—non-reactive container; (107B)—nozzle; (108)—Blower; (109)—Outlet; (700A), (700B), (700C)—method including steps; 800A)—DCD mechanism step 1; (800B)—DCD mechanism step 2; (800C)—DCD mechanism step 3.

### DESCRIPTION OF THE INVENTION

In the following description, for the purpose of explanation, specific details are set forth in order to provide an understanding of the invention. It will be device/system, however, to one skilled in the art that the invention may be practiced without these details. One skilled in the art will recognize that embodiments of the present invention, some of which are described below, may be incorporated into a number of different purifier devices/systems. Structures and devices/system shown in the block diagram/s are illustrative of exemplary embodiments of the invention and are meant to avoid obscuring the invention. Furthermore, connections between components within the figures are not intended to be limited to direct connections.

Reference in the specification to “one embodiment” or “an embodiment” means that a particular feature, structure, characteristic, or function described in connection with the embodiment is included in at least one embodiment of the invention. The appearances of the phrase “in one embodiment” in various places in the specification are not necessarily all referring to the same embodiment. The headings and subheadings used in the document are not intended to limit the content therein to the respective heading or subheading. Rather, they are used to help readers navigate and prevent obscuring the invention.

Further, the disclosure refers to purification to include various aspects including but not limited to sterilization, disinfection, sanitization, removal of particulate matter and harmful gases.

In various embodiments herein, a self-cleaning air purifier is disclosed. The system (100) may comprise of a UV source (102) configured to produce irradiation to effect microorganisms/pathogens; an ionizer (104) configured to generate dense electric field and thus produce OH radicals and/or H202 using the energy imparted by the UV source (102) and a water source; a filter (106) having an inner surface and an outer surface, configured to trap impurities of the impure air and produce filtered air from the outer surface; wherein the UV source (102) and ionizer (104) are configured to face the inflow of impure air before the impure air reaches the filter (106).

The inlet (101) is placed longitudinally towards the center of the housing, at a specified distance from the bottom; at least an electrical compartment (103), a photocatalytic coating (105) on inside the filter (106) assembly; with at least a chemical oxidant system (107), with at least a blower (108), and with at least an outlet (109); at least one needle (104A) and an chamber (104B) for placing objects and/or belongings therein for disinfection. The present invention is directed towards a self-cleaning air purifier (100), having enhanced safety levels by keeping the inner and outer body of the device sterilized (air, surface, belongings) continuously. The system (107) may be used for surface sterilization by aerosolizing chemical oxidation of known amount depending on the room size.

The present invention is also disclosing a method comprising the steps of first, subjecting the impure air to UV source causing inflight deactivation and surface deactivation of microorganisms in the impure air. Second, subjecting the impurities in the impure air to electrons produced by ionizer, thereby producing conductive and inductive forces for increased filtration by having impurities stick to the filter, and produce OH radicals using the energy imparted by the UV source and a water source, further causing inflight deactivation and surface deactivation of microorganisms in the impure air. Third, subjecting the impure air to a filter having an inner surface and an outer surface, configured to trap impurities of the impure air and produce filtered air from the outer surface, wherein the directing of air causes impure air to be subject to UV source and ionizer before the impure air reaches the filter.

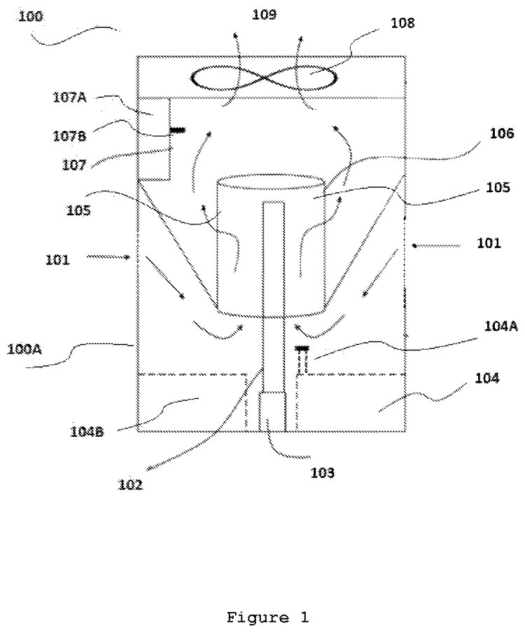

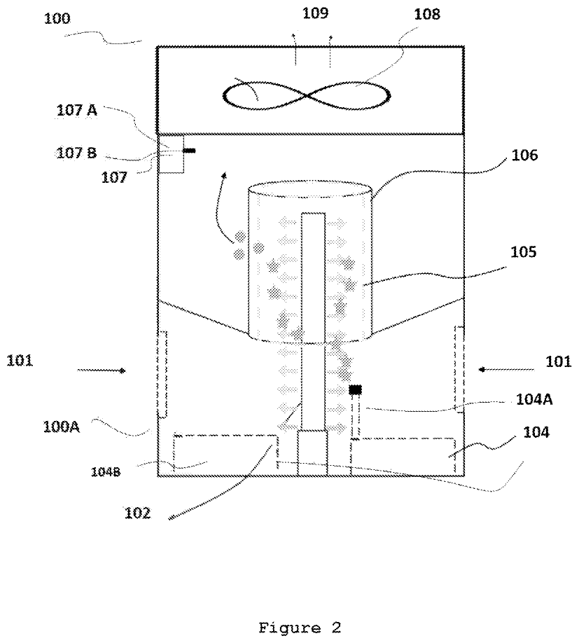

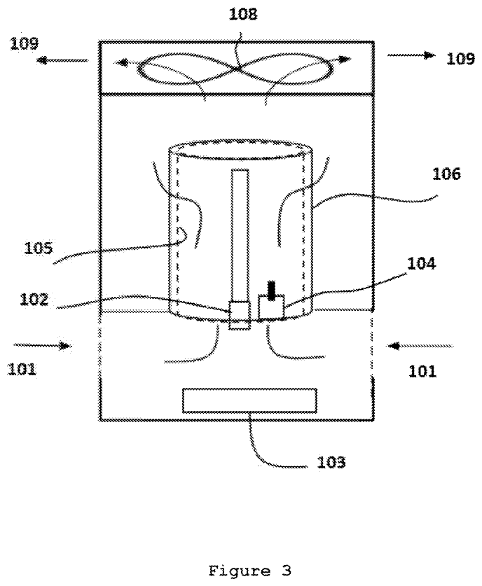

FIG. 1, FIG. 2, and FIG. 3 may be referred to for understanding various aspects explained below Impure air may be let into the system through an inlet (101) which is preferably placed longitudinally towards the center of the housing (100A), at a specified distance from the bottom, such that large-sized particles can not enter in the purifier/system (100). This aids in increasing the lifetime of the filter being used and also in improving the disinfection efficiency. The impure air inlet and the outlet for purified air may be configured to allow for batch processing of the air. Further, in another embodiment the impure air inlet and the outlet for purified air may be configured to allow for continuous processing of the air.

The airflow is created by means of a blower (108) or a fan and has a rotatable head, thereby enabling adjustable/directional flow of air. This enables the sanitized air to be directed onto a person or object for a predetermined time to ensure one's safety against pathogens by the virtue of the direction of the air stream.

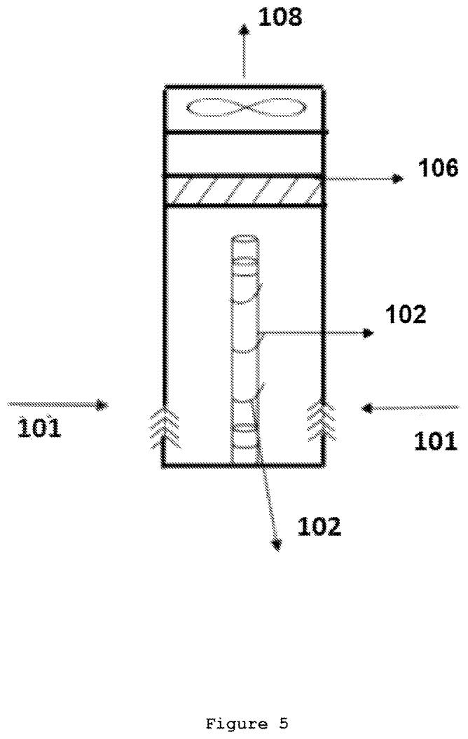

The UV light source (102) is preferably positioned longitudinally to the flow of air ensuring continuous disinfection of the airstream due to increased residence time. The filter (106) is preferably positioned surrounding/around the UV source (102), whereby the UV rays continuously irradiate onto the inner side of filter (106) to kill microorganisms trapped therein and disinfect the filter (106). This aids in inactivating all kinds of the pathogen on filter (106) and the user won't be infected while handling the air purifier/device (100) (e.g., while filter replacement, repairing, etc). The UV source (102) is being cleaned by using electrostatic fields. The outlet grills/housing is designed in such a way that would avoid a direct eye contact with the UV source (102) in various configurations. In one embodiment, the UV source may be cylindrical. Further, the UV source may be configured to irradiate with wavelengths between 100 and 400 nm. In another embodiment, the UV source may also be flat as shown in FIG. 5.

In an embodiment, an ionizer may be configured to generate dense electric fields and thus produce OH radicals and/or hydrogen peroxide using the energy imparted by the UV source and a water source. The Ionizer is configured to induce conductive and inductive forces for increased filtration. The ionizer is configured to generate anions to cause self cleaning of the UV source from the dust particles. Carbon fibers from the ionizer (104) which operates at a very high voltage are used to remove all kinds of deposited contaminants from the UV surface. The carbon fibers operating at high voltage also help in the formation of OH radicals using H20 molecules in the ambient air which is a powerful oxidant and kills pathogens. Alternatively, needles(104A) of the ionizer are used to create electric fields. Thus, the carbon fibers and/or needles (104A) act as a secondary source of disinfection.

The filter (106) is positioned such that air flows from the inner side to the outer side. The filter(106) is preferably UV resistant, with the inner side coated with a photocatalyst (105) selected from, but not limited to, Ti02, cuprous oxide, zinc oxide, and silicon dioxide which acts as an extra layer of sanitization for better disinfection through the process of photocatalysis. The filter(106) can also be impregnated with silver nanoparticles (or any other material which has antimicrobial properties) for further sanitization and disinfection effect. The filter (106) further has the outer side coated with an adsorbent (e.g. activated carbon) to trap gases and VOCs (volatile organic compounds). The inner surface of the filter that faces inflow of the air may be configured to be facing the UV source. Furthermore, the inner surface of the filter that faces inflow of the air is also facing the ionizer. Furthermore, the outer surface of the filter is coated with an adsorbent to trap gases and VOCs. In one exemplary embodiment, the adsorbent may be activated carbon.

The filter is cylindrical in shape engulfing said UV source. This may allow for increased interaction of the UV source and the filter surface. In another embodiment, the filter may be flat in shape. This may provide for easier manufacturability. A mesh towards the inner surface of the filter having photocatalyst coating is provided for increased effect on microorganisms/pathogens. An anti microbial coating on the filter may be provided to affect the microorganism that are trapped in the filter.

In an embodiment herein, a chemical oxidation system for introducing chemical oxidation agent into the filtered air after the filtration may also be provided. The Chemical oxidant (107) may use chemicals such as hydrogen peroxide etc., which acts as an on-demand disinfection system. The integrated chemical fogging system (107) comprising the chemical oxidant disinfects the air as well as the surface in a closed space/room. The oxidant (107) is stored in a non-reactive container (107A) inside the housing optionally with a pump for atomization through the nozzle (107B). The amount of chemical oxidant released in a specific amount of time may be controlled using a valve.

In an embodiment, the disinfecting chamber (104B) uses UV rays for the disinfection of small objects/items. Extra UV sources (102) may be included in these chambers (104B) if required. The walls of this chamber (104B) may be coated with a material that reflects maximum UV light, such as dispersion of barium sulfate in an aqueous solution of a water-soluble inorganic binder. The chamber (104B) is being used as a sterilization chamber (104B) for objects like mobiles, wallets etc.

In an embodiment, the self-cleaning air-purifier (100) includes at least an electrical compartment (103) having a PCB ballast, which gives an appropriate power supply as well as controls the power supply to the UV (102). A sensor may also be provided such that it detects movement and automatically switches the device ON/OFF.

A person skilled in the art may realize that various embodiments may be used in an closed inhouse environment as well as external environments with such customizations to suit the requirement.

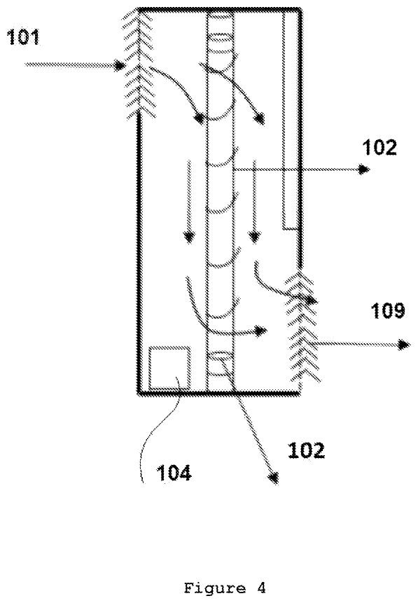

The present invention as illustrated in FIG. 4 provides another embodiment of a self-cleaning air-purifier (100) comprising a housing (100A) having at least one inlet (101), at least one UV source (102) comprising a UV ballast and UV, at least one filter (106), which coated with a photocatalyst (105) and at least one outlet (109). A self-cleaning air-purifier (100) designed for a duct with some flow rate at low pressure such as for cars. This may be more useful in situations where sterilization (microbial deactivation) is required, not filtration (air purification). The air flow may be configured specially for cars because of restrictions in longitudinal direction.

In an embodiment, the photocatalyst (105) is coated preferably on both the inner and outer body of the housing (100A). The inner coating will enhance the process of disinfection by the production of OH radicals. The outer coating of the photocatalyst will ensure the surface of the air-purifier/device (100) remains pathogen-free. The photocatalyst (105) is selected from Titanium oxide, cuprous oxide, zinc oxide, and silicon dioxide which acts as an extra layer of sanitization for better disinfection and removes VOCs through the process of photocatalytic degradation. The purifier may be provided to have a small, compact size, and due to which it can be used to sanitize small, enclosed spaces like cars, cabins, etc.

The present invention as illustrated in FIG. 5 another embodiment of a self-cleaning air-purifier (100) comprising a filter (106) is placed above the UV source (102) and the air after irradiation with the UV rays passes through the flat filter (106) before moving towards the outlet (109).

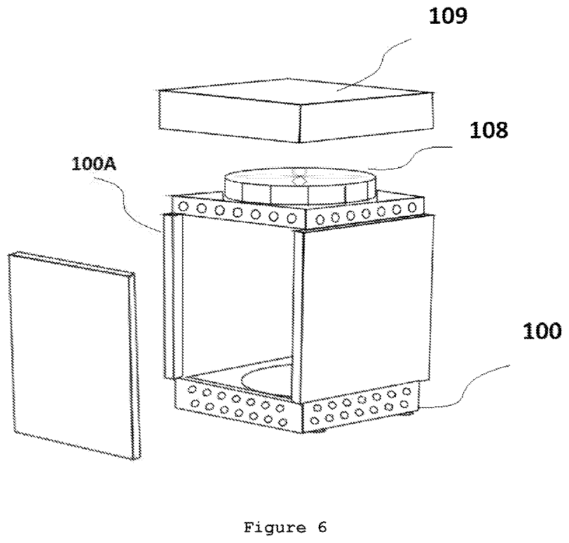

FIG. 6 illustrates a housing of the air purifier as per an embodiment herein. As may be seen, a housing (100A), may be provided to have a cubical shape. Further, air ventilation holes may be provided on the upper and lower ends thereof. Furthermore, a blower (108) may be provided on the upper end of the said cubical shape sitting next to an outlet (109).

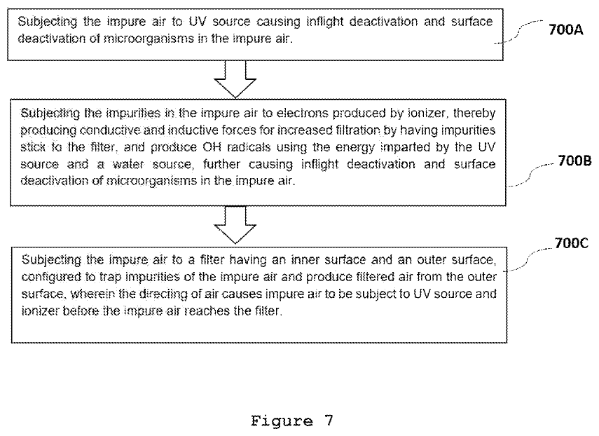

The present invention as illustrated in FIG. 7 another embodiment invention provides a method of air purification comprising the steps of: step 1 (700A), subjecting the impure air to UV source causing inflight deactivation and surface deactivation of microorganisms in the impure air. Step 2 (700B), subjecting the impurities in the impure air to electrons produced by ionizer, thereby producing conductive and inductive forces for increased filtration by having impurities stick to the filter, and produce OH radicals using the energy imparted by the UV source and a water source, further causing inflight deactivation and surface deactivation of microorganisms in the impure air. Step 3 (700C) subjecting the impure air to a filter having an inner surface and an outer surface, configured to trap impurities of the impure air and produce filtered air from the outer surface, wherein the directing of air causes impure air to be subject to UV source and ionizer before the impure air reaches the filter.

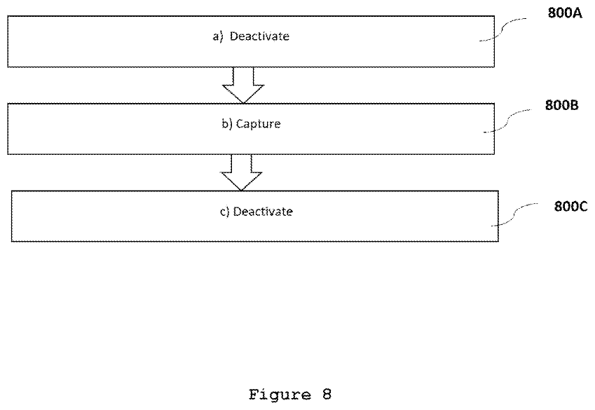

The present invention as illustrated in FIG. 8 another embodiment invention provides DCD (deactivate, Capture, Deactivate) approach/mechanism comprising steps of: Step 1 (800A): Deactivation includes i) The germs undergo inflight deactivation by germicidal rays and OH radicals as they travel along with the air purifier; ii) The continuous irradiation of germicidal rays and continuous bombardment of anions formed due to electromigration and OH radicals formed by ionization and photocatalysis deactivates the germs; iii) The air particles have a much higher residence time for inflight deactivation and a configuration that provides the highest possible intensity of germicidal rays and OH radicals. Step 2 (800B): Capture: includes i) The filters (106) are used to trap viable and non-viable particulate matter; the filters can be coated with antimicrobial coating for deactivation of germs at the surface level. Step 3 (800C): Deactivation: includes the captured viable particulate matter or germs that are deactivated through the prolonged exposure to germicidal rays and continuous bombardment of OH radicals and anions formed in the process of ionization and on photocatalyst coated surfaces.

In another aspect, purifier housing receives air from the inlet. Further, letting the air stream pass through the UV source, leads to inflight deactivation of microorganisms. Further, letting the air stream also pass through the Ionizer, leads to inflight deactivation of microorganisms due to OH radicals formed by ionization. Further, ionizer removes deposited dust on the UV source and maintains high disinfection efficiency of the device. Ionizers induce conductive and inductive forces for higher filtration efficiency. Further, a photocatalyst and/or anti-microbial coating of the filter acts as an extra layer of sterilization. External mesh is also used for photocatalyst coating to maximize the surface area. Further aspect includes the continuous irradiation to OH radicals formed in the process of photocatalyst and ionization on the filter to continuously deactivate captured microorganisms. The continuous UV source irradiations on the filter (106) leads to continuous deactivation of microorganisms. Step 10: The filter outer side which is coated with an adsorbent that traps gases and VOCs. Further, an aspect includes directing the air toward the outlet using a blower.

Advantageously, the present invention provides a self-cleaning air purifier (100) utilizing integrated technology of UV radiation, photocatalysis, filtration, coatings and disinfectant spray in a unique and novel compact arrangement for removal of airborne infection while simultaneously disinfecting small personal belongings.

Unlike contemporary air purification devices, the present invention has the provision to holistically approach various aspects of air purification. More particularly, particulate matter either viable or non-viable will be removed using the present invention device. The present invention self-cleaning air purifier/device provides filtration, disinfection, and sterilization of air streams as a complete solution to the problems of air pollution and disease spread. Also, dust and other particulate matter that gets deposited on the surface of the UV source which may affect the efficiency of the purifier/system over time is overcome using the self-cleaning approach hence creating automatic cleaning and maintenance, thereby improving efficiency.

Furthermore, Unlike pre existing devices in contemporary art, in the present invention the purifier/device remains sterilized such that overuse of filters doesn't make the purifier/device a source of microbial contamination. UV source has a longitudinal arrangement between the air stream and UV light to significantly increase the residence time for better disinfection.

The foregoing description of the invention has been set merely to illustrate the invention and is not intended to be limiting. Since the modifications of the disclosed embodiments incorporating the spirit and substance of the invention may occur to the person skilled in the art, the invention should be construed to include everything within the scope of the disclosure and claims appended.

We claim:

1. An air purification system, for purifying incoming impure air, the system comprising:
a UV source configured to produce irradiation to effect microorganisms;
an ionizer configured to emit electrons and thus produce OH radicals using the energy imparted by the UV source and a water source;
a filter having an inner surface and an outer surface, configured to trap impurities of the impure air and produce filtered air from the outer surface;
wherein the UV source and ionizer are configured to face the inflow of impure air before the impure air reaches the filter.

2. An air purification system as in claim 1, wherein the ionizer is configured to emit electrons and thus produce hydrogen peroxide using the energy imparted by the UV source and a water source.

3. An air purification system as in claim 1, wherein the water source is the humidity in the air.

4. An air purification system as in claim 1, comprising a chemical oxidation system for introducing chemical oxidation agent into the filtered air after the filtration.

5. An air purification system as in claim 1, wherein the Ionizer is configured to impart charges on impurities thereby producing conductive and inductive forces for increased filtration by having impurities stick to the filter.

6. An air purification system as in claim 1, ionizer configured to generate anions to cause auto-cleaning of the UV source from the dust particles.

7. An air purification system as in claim 1, wherein the inner surface of the filter that faces inflow of the air is facing the UV source for in-situ deactivation of microorganism.

8. An air purification system as in claim 1, wherein the inner surface of the filter that faces inflow of the air is facing the ionizer.

9. An air purification system as in claim 1, wherein the inflow of the impure air is parallel to the surface of the UV source.

10. An air purification system as in claim 1, wherein the outer surface of the filter is coated with an adsorbent to trap gases and volatile organic compounds.

11. An air purification system as in claim 10, wherein the adsorbent is activated carbon.

12. An air purification system as in claim 1, comprising a mesh towards the inner surface of the filter having photocatalyst coating for increased effect on microorganisms.

13. An air purification system as in claim 1, comprising: an anti microbial coating on the filter.

14. An air purification system as in claim 1, wherein UV source is cylindrical.

15. An air purification system as in claim 1, wherein the UV source is configured to irradiate with wavelengths between 100 and 400 nm.

16. An air purification system as in claim 1, wherein the filter is cylindrical in shape engulfing said UV source.

17. An air purification system as in claim 1, wherein the filter is flat in shape.

18. An air purification system as in claim 1, wherein the impure air inlet and the outlet for purified air are configured to allow for batch processing of the air.

19. An air purification system as in claim 1, wherein the impure air inlet and the outlet for purified air are configured to allow for continuous processing of the air.

20. An air purification system, for purifying an incoming impure air, the system comprising:
a housing;
an inlet for impure air;
an adjustable outlet for purified air;
a UV source configured to produce irradiation to effect microorganisms/pathogens;
an ionizer configured to generate dense electric field; a filter having an inner surface and an outer surface, configured to trap impurities of the impure air and produce filtered air from the outer surface;
wherein the inner surface of the filter facing the incoming impure air also faces the UV source and ionizer thus providing self sterilization/cleaning of the filter.

21. An air purification system as claimed in claim 20, wherein the air outlet is configured to allow air flow in opposite direction for inducing self cleaning of the filter using chemical oxidation agent.

22. An air purification system as claimed in claim 20, wherein the adjustable outlet is configured to direct air using flaps and blades.

23. A method for purifying incoming impure air containing impurities, the steps comprising:
Subjecting the impure air to UV source causing inflight deactivation and surface deactivation of microorganisms in the impure air;
Subjecting the impurities in the impure air to electrons produced by ionizer, thereby producing conductive and inductive forces for increased filtration by having impurities stick to the filter, and produce OH radicals using the energy imparted by the UV source and a water source, further causing inflight deactivation and surface deactivation of microorganisms in the impure air;
Subjecting the impure air to a filter having an inner surface and an outer surface, configured to trap impurities of the impure air and produce filtered air from the outer surface, wherein the directing of air causes impure air to be subject to UV source and ionizer before the impure air reaches the filter.

24. A method for purifying incoming impure air containing impurities, as claimed in claim 23, further comprising: directing the air after purification towards a user using a blower.
